(19) United States
(12) Patent Application Publication
(10) Pub. No.: US 2026/0268517 A1
(43) Pub. Date: Sep. 10, 2026

(54) MEDICAL IMAGE DIAGNOSTIC APPARATUS AND MEDICAL IMAGE PROCESSING METHOD

(71) Applicant: CANON KABUSHIKI KAISHA, Tokyo (JP)

(72) Inventors: Shuki MARUYAMA, Nasushiobara Tochigi (JP); Hidenori TAKESHIMA, Taito Tokyo (JP)

(73) Assignee: CANON KABUSHIKI KAISHA, Tokyo (JP)

(21) Appl. No.: 19/432,260

(22) Filed: Dec. 24, 2025

(30) Foreign Application Priority Data
Mar. 4, 2025 (JP) 2025-033470

(51) Int. Cl. G06T 7/73; A61B 5/00; G06T 7/11; G16H 50/20

(52) U.S. Cl. CPC G06T 7/73; A61B 5/4561; A61B 5/7425; G06T 7/11; G16H 50/20; G06T 2207/20081; G06T 2207/20084; G06T 2207/20221; G06T 2207/30008

(57) ABSTRACT

A medical image diagnostic apparatus includes processing circuitry configured to acquire an optical image of a subject captured by an optical imaging device and/or skeleton information about the subject, generate a medical image related to the subject based on the optical image and/or the skeleton information, and cause a display to display the medical image.

### CROSS-REFERENCE TO RELATED APPLICATIONS

This application is based upon and claims the benefit of priority from Japanese Patent Application No. 2025-033470, filed on Mar. 4, 2025, the entire contents of which are incorporated herein by reference.

### FIELD

Embodiments described herein relate generally to a medical image diagnostic apparatus and a medical image processing method.

### BACKGROUND

Some medical image diagnostic apparatuses of related art are configured to perform imaging for positioning verification after visually observing a position and posture of a subject or checking the position and posture with a camera. For example, in a magnetic resonance imaging (MRI) apparatus and an X-ray computed tomography (CT) apparatus, positioning imaging (also referred to as positioning scan, prescan, locator imaging, scanography, or the like) is generally executed first. Specifically, in an MRI apparatus and an X-ray CT apparatus, positioning imaging is executed prior to main imaging (also referred to as main scan). Further, in an X-ray diagnostic apparatus (e.g., an angiographic apparatus), fluoroscopic imaging can be performed prior to direct imaging (e.g., chest X-ray imaging).

MRI apparatuses of related art include a positioning support function that uses an optical camera. The positioning support function is a function for, for example, estimating skeleton information about a subject based on a camera image captured by the optical camera and supporting positioning for main imaging by the MRI apparatus. In this case, the positioning support function supports the layout of a coil to be used for main imaging by, for example, displaying a position of a projector provided on the ceiling of an examination room in which the MRI apparatus is placed.

In any of the above-described cases, it is necessary to perform imaging (positioning imaging) for positioning verification of a subject prior to main imaging in regard to an imaging position or the like in main imaging.

### BRIEF DESCRIPTION OF THE DRAWINGS

FIG. 1 is a block diagram illustrating a configuration of a magnetic resonance imaging (MRI) apparatus according to an embodiment;

FIG. 2 illustrates an example of generation of a medical image according to the embodiment;

FIG. 3 illustrates an example of generation of the medical image by using skeleton information according to the embodiment;

FIG. 4 illustrates an example of generation of the medical image by using an optical image and skeleton information according to the embodiment;

FIG. 5 illustrates an example of generation of the medical image by further using a breathing condition according to the embodiment;

FIG. 6 illustrates an example of generation of the medical image corresponding to a segmentation image according to the embodiment;

FIG. 7 illustrates an example of generation of the medical image based on a previous optical image, a previous main captured image, and a current optical image according to the embodiment;

FIG. 8 illustrates an example of determination of a posture state of a subject by applying the medical image to a posture output learned model according to the embodiment;

FIG. 9 illustrates an example of a display screen to select imaging (scan) for the subject according to the embodiment;

FIG. 10 illustrates an example of a display screen to select imaging (scanning) for the subject according to the embodiment when an X-ray computed tomography (CT) apparatus is used as a medical image diagnostic apparatus;

FIG. 11 illustrates an example of a display screen to select imaging for the subject according to the embodiment when an X-ray diagnostic apparatus is used as the medical image diagnostic apparatus;

FIG. 12 illustrates an example of a medical image to be displayed on a display provided on a gantry according to the embodiment;

FIG. 13 illustrates an example of a projector position superimposed on a medical image according to the embodiment;

FIG. 14 illustrates an example where a centerline of a vertebral body is superimposed on an optical image on the display provided on the gantry according to the embodiment;

FIG. 15 illustrates an example where a projector position and a main imaging range are superimposed on a medical image displayed on the display according to the embodiment;

FIG. 16 illustrates an example where a plurality of main imaging ranges corresponding to long length imaging and a projector position are superimposed on a medical image displayed on the display according to the embodiment;

FIG. 17 illustrates an example of adjustment of a main imaging range according to the embodiment;

FIG. 18 illustrates an example where a projector position is automatically moved according to the embodiment; and

FIG. 19 is a flowchart illustrating an example of a non-imaging positioning processing procedure according to the embodiment.

### DETAILED DESCRIPTION

A medical image diagnostic apparatus according to an embodiment includes processing circuitry. The processing circuitry acquires an optical image of a subject captured by an optical imaging device and/or skeleton information about the subject. The processing circuitry generates a medical image related to the subject based on the optical image and/or the skeleton information. The processing circuitry causes a display to display the medical image.

Embodiments of a medical image diagnostic apparatus and a medical image processing method will be described below with reference to the drawings. For specific description, it is assumed that a magnetic resonance imaging (MRI) apparatus is used as the medical image diagnostic apparatus. The medical image diagnostic apparatus according to the embodiment is not limited to an MRI apparatus, but instead may be implemented as for example, an X-ray computed tomography (CT) apparatus, an X-ray diagnostic apparatus, a positron emission tomography (PET) apparatus, or a single photon emission computed tomography (SPECT).

More alternatively, the medical image diagnostic apparatus may be implemented as a composite apparatus such as a PET-CT apparatus, an SPECT-CT apparatus, a PET-MRI apparatus, or an SPECT-MR apparatus. In the following embodiments, it is assumed that parts denoted by the same reference numerals perform similar operation, and repeated description is omitted as appropriate.

Various Embodiments will be described hereinafter with reference to the accompanying drawings.

### Embodiments

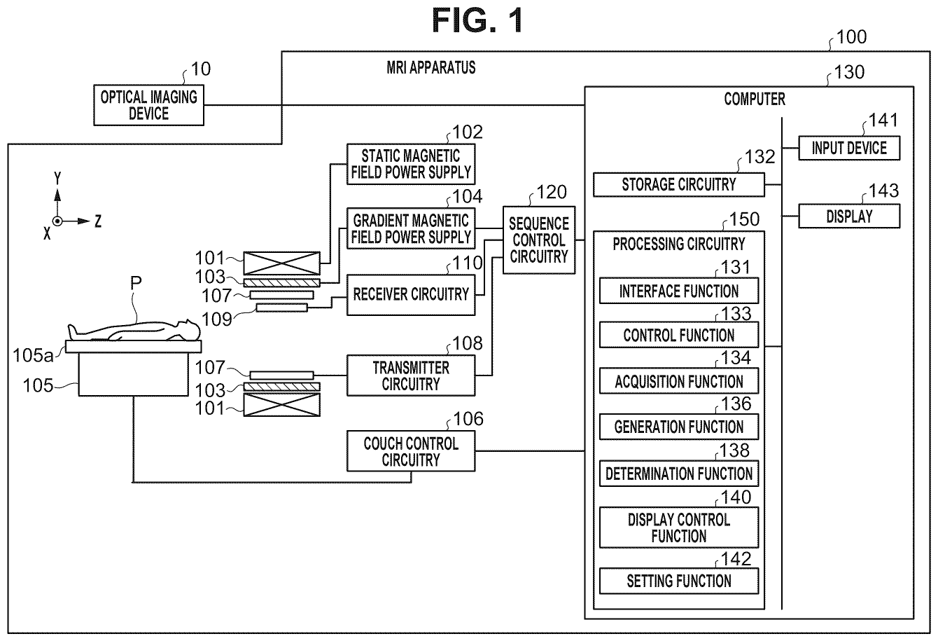

FIG. 1 is a block diagram illustrating a configuration of an MRI apparatus 100 according to an embodiment. As illustrated in FIG. 1, the MRI apparatus 100 includes a static field magnet 101, a static magnetic field power supply 102, a gradient coil 103, a gradient magnetic field power supply 104, a couch 105, couch control circuitry 106, a transmitter coil 107, transmitter circuitry 108, a receiver coil 109, receiver circuitry 110, sequence control circuitry 120, and a computer 130 (also referred to as a medical image processing apparatus). The MRI apparatus 100 does not include a subject P (e.g., a human body). The configuration illustrated in FIG. 1 is merely an example. For example, the units in each of the sequence control circuitry 120 and the computer 130 may be integrated together or separated from each other, as appropriate. A gantry in the MRI apparatus 100 includes, for example, the static field magnet 101, the static magnetic field power supply 102, the gradient coil 103, the gradient magnetic field power supply 104, the transmitter coil 107, the transmitter circuitry 108, the receiver coil 109, the receiver circuitry 110, and the sequence control circuitry 120. The gantry may be provided with a display 143.

As illustrated in FIG. 1, an optical imaging device 10 is connected to the MRI apparatus 100 by a known method such as a wired connection or a wireless connection. The optical imaging device 10 is provided on, for example, a ceiling of an examination room in which the MRI apparatus 100 is placed. Specifically, the optical imaging device 10 is placed on the ceiling immediately above the couch 105 in the MRI apparatus 100. The location where the optical imaging device 10 is placed is not limited to the ceiling of the examination room. The optical imaging device 10 can be placed at any location as long as an image of the entire area of a couchtop 105a can be captured.

The optical imaging device 10 captures an optical image of an object included in an imaging range of the optical imaging device 10 constantly or in response to a user instruction. The optical imaging device 10 generates an image obtained by capturing an object included in the imaging range of the optical imaging device 10 (this image is hereinafter referred to as an optical image). The optical image can be obtained regardless of the presence or absence of coils and fastening devices. A neural network model to which an optical image and/or skeleton information about the subject P is input is generated so as to generate not only the optical image obtained before coils are arranged, but also the optical image obtained after coils are arranged. A learned neural network model (learned model) will be described below.

The optical imaging device 10 transmits the optical image to the MRI apparatus 100. The optical imaging device 10 corresponds to, for example, an optical camera. The optical image may also be referred to as a camera image. In the example illustrated in FIG. 1, the optical imaging device 10 is illustrated as a device separated from the MRI apparatus 100, but instead may be included in the configuration of the MRI apparatus 100. Any known device can be used as the optical imaging device 10, and thus the description thereof is omitted.

The static field magnet 101 is a magnet that is formed in a substantially cylindrical hollow shape and generates a static magnetic field in an internal space. The static field magnet 101 is, for example, a superconducting magnet, and receives a current supplied from the static magnetic field power supply 102 and is excited by the current. The static magnetic field power supply 102 supplies a current to the static field magnet 101. The static field magnet 101 may be a permanent magnet. In this case, the MRI apparatus 100 need not necessarily include the static magnetic field power supply 102. The static magnetic field power supply 102 may be provided separately from the MRI apparatus 100.

The gradient coil 103 is a coil that is formed in a substantially cylindrical hollow shape and is located on the inside of the static field magnet 101. The gradient coil 103 is formed by combining three coils corresponding to an X-axis, a Y-axis, and a Z-axis, respectively, which are orthogonal to each other. The three coils individually receive currents supplied from the gradient magnetic field power supply 104, and generate gradient magnetic fields with magnetic field strengths that vary along the X-axis, the Y-axis, and the Z-axis, respectively. The gradient magnetic fields generated by the gradient coil 103 along the X-axis, the Y-axis, and the Z-axis correspond to, for example, a slicing gradient magnetic field Gs, a phase encoding gradient magnetic field Ge, and a readout gradient magnetic field Gr, respectively. The gradient magnetic field power supply 104 supplies a current to the gradient coil 103.

The couch 105 includes the couchtop 105a on which the subject P is placed. Under the control of the couch control circuitry 106, the couchtop 105a is inserted into a cavity (imaging port) of the gradient coil 103 in a state where the subject P is placed. In general, the couch 105 is placed in such a manner that the longitudinal direction of the couch 105 is parallel to the central axis of the static field magnet 101. The couch control circuitry 106 drives the couch 105 to move the couchtop 105a in the longitudinal direction and an up-down direction under the control of the computer 130.

The transmitter coil 107 is located on the inside of the gradient coil 103, and receives a radio-frequency (RF) pulse supplied from the transmitter circuitry 108 and generates a high-frequency magnetic field. The transmitter circuitry 108 supplies, to the transmitter coil 107, the RF pulse corresponding to a Larmor frequency determined depending on the type of a target atom and a magnetic field strength.

The receiver coil 109 is located on the inside of the gradient coil 103, and receives a magnetic resonance (MR) signal emitted from the subject P due to the effect of the high-frequency magnetic field. Upon receiving the MR signal, the receiver coil 109 outputs the received MR signal to the receiver circuitry 110.

The transmitter coil 107 and the receiver coil 109 described above are merely examples. The transmitter coil 107 and the receiver coil 109 may be configured using a coil including only a transmission function, a coil including only a reception function, or a coil including the transmission and reception functions, or may be configured by combining one or more of the coils.

The receiver circuitry 110 detects the MR signal output from the receiver coil 109 and generates MR data based on the detected MR signal. Specifically, the receiver circuitry 110 generates MR data by converting the MR signal output from the receiver coil 109 into a digital signal. The receiver circuitry 110 transmits the generated MR data to the sequence control circuitry 120. The receiver circuitry 110 may be provided on a gantry apparatus including the static field magnet 101 and the gradient coil 103.

The sequence control circuitry 120 drives each of the gradient magnetic field power supply 104, the transmitter circuitry 108, and the receiver circuitry 110 based on sequence information transmitted from the computer 130, to thereby perform imaging of the subject P. In this case, the sequence information is information that defines a procedure for imaging and is also referred to as a sequence condition. The sequence information defines the intensity of a current to be supplied from the gradient magnetic field power supply 104 to the gradient coil 103, a timing for supplying the current, the intensity of an RF pulse to be supplied from the transmitter circuitry 108 to the transmitter coil 107, a timing for applying the RF pulse, a timing for the receiver circuitry 110 to detect an MR signal, and the like.

For example, the sequence control circuitry 120 is an integrated circuit such as an application specific integrated circuit (ASIC) or a field programmable gate array (FPGA), or electronic circuitry such as a central processing unit (CPU) or a micro processing unit (MPU). The sequence control circuitry 120 corresponds to a sequence control unit and may also be referred to as an imaging unit. The imaging unit may correspond to the gantry in the MRI apparatus 100.

Upon receiving MR data from the receiver circuitry 110 as a result of driving each of the gradient magnetic field power supply 104, the transmitter circuitry 108, and the receiver circuitry 110 to perform imaging of the subject P, the sequence control circuitry 120 transfers the received MR data to the computer 130.

The computer 130 controls the overall operation of the MRI apparatus 100 and performs image generation processing and the like. The computer 130 includes a storage circuitry 132, an input device 141, the display 143, and processing circuitry 150. The processing circuitry 150 includes an interface function 131, a control function 133, an acquisition function 134, a generation function 136, a determination function 138, a display control function 140, and a setting function 142.

Each processing function to be implemented by the interface function 131, the control function 133, the acquisition function 134, the generation function 136, the determination function 138, the display control function 140, and the setting function 142 is stored in the storage circuitry 132 in the form of a program that can be executed by the computer 130. The processing circuitry 150 reads out programs from the storage circuitry 132 and executes the programs to thereby implement the functions corresponding to the programs. In other words, the processing circuitry 150 that has read out programs includes the corresponding functions in the processing circuitry 150 illustrated in FIG. 1.

While FIG. 1 illustrates an example where a single processing circuitry 150 implements processing functions to be performed by the interface function 131, the control function 133, the acquisition function 134, the generation function 136, the determination function 138, the display control function 140, and the setting function 142, the processing circuitry 150 may be configured by combining a plurality of independent processors and each processor may execute programs to implement the corresponding functions. In other words, each of the functions described above may be configured as a program and a single processing circuitry 150 may execute each program, or a specific function may be implemented on dedicated independent program execution circuitry.

The term “processor” used in the above description refers to, for example, a CPU, a graphical processing unit (GPU), or circuitry such as an application specific integrated circuit or a programmable logic device (e.g., a simple programmable logic device (SPLD), a complex programmable logic device (CPLD), and an FPGA). The processor reads out programs stored in the storage circuitry 132 and executes the programs to thereby implement the corresponding functions.

Instead of storing programs in the storage circuitry 132, programs may be directly incorporated in the circuitry of the processor. In this case, the processor reads out programs incorporated in the circuitry and executes the programs to thereby implement the corresponding functions. The couch control circuitry 106, the transmitter circuitry 108, the receiver circuitry 110, and the like are also configured using electronic circuitry such as the processor as described above.

The processing circuitry 150 causes the interface function 131 to transmit sequence information to the sequence control circuitry 120 and receive MR data from the sequence control circuitry 120. Upon receiving MR data, the processing circuitry 150 including the interface function 131 stores the received MR data in the storage circuitry 132. The processing circuitry 150 that implements the interface function 131 corresponds to an interface unit. Other functions in the processing circuitry 150 will be described below.

The storage circuitry 132 stores MR data received by the processing circuitry 150 including the interface function 131, various types of data acquired by the acquisition function 134, various types of image data generated by the generation function 136, determination results obtained by the determination function 138, various types of data to be displayed by the display control function 140, various types of data set by the setting function 142, and the like. Further, the storage circuitry 132 stores MR data located in a k-space by the control function 133 (this data is also referred to as k-space data). The various types of data to be stored will be described below. For example, the storage circuitry 132 is implemented as a semiconductor memory element such as a random access memory (RAM) or a flash memory, a hard disk, an optical disk, or the like. The storage circuitry 132 is an example of a storage unit or a memory.

The input device 141 receives various types of instructions and information input from a user. The input device 141 is implemented as, for example, a trackball, a switch button, a mouse, a keyboard, a touch-pad used to perform an input operation in response to a touch on an operation surface, a touch screen having a configuration in which a display screen and a touch-pad are integrated, non-contact input circuitry using an optical sensor, audio input circuitry, and the like. The input device 141 is electrically connected to the processing circuitry 150. The input device 141 converts an input operation received from an operator into an electric signal, and outputs the electric signal to the processing circuitry 150. The input device 141 is an example of an input unit or an operation unit.

The input device 141 used herein is not limited only to an input device including a physical operation component (input interface) such as a mouse and a keyboard. Examples of the input device 141 also include electric signal processing circuitry that receives an electric signal corresponding to an input operation from an external input device provided separately from the MRI apparatus 100 and outputs the electric signal to control circuitry. The input device 141 is not limited only to an input device mounted on the computer 130, but instead may be mounted on, for example, the gantry.

The display 143 displays a graphical user interface (GUI) for receiving an input of imaging conditions, images generated by the generation function 136 of the processing circuitry 150, and the like under the control of the processing circuitry 150 including the control function 133. The display 143 is implemented as, for example, a cathode-ray tube (CRT) display, a liquid crystal display, an organic electroluminescence (EL) display, a light-emitting diode (LED) display, a plasma display, any other display known in this technical field, or a display device such as a monitor. The display 143 is an example of a display unit.

The processing circuitry 150 causes the control function 133 to control the overall operation of the MRI apparatus 100 to control imaging, image generation, image display, and the like. For example, the processing circuitry 150 including the control function 133 receives an input of imaging conditions (imaging parameters and the like) on the GUI, and generates sequence information according to the received imaging conditions. The processing circuitry 150 including the control function 133 transmits the generated sequence information to the sequence control circuitry 120. The processing circuitry 150 that implements the control function 133 corresponds to a control unit. The processing circuitry 150 that implements the control function 133 is an example of the control unit.

The processing circuitry 150 causes the acquisition function 134 to acquire an optical image of the subject P captured by the optical imaging device 10 and/or skeleton information about the subject P. In other words, the acquisition function 134 acquires an optical image from the optical imaging device 10 by imaging using the optical imaging device 10. The skeleton information about the subject P is generated by the generation function 136 using a known method. In this case, the acquisition function 134 acquires the skeleton information from the generation function 136. The acquisition function 134 causes the storage circuitry 132 to store the acquired optical image or skeleton information. The processing circuitry 150 that implements the acquisition function 134 is an example of an acquisition unit.

The processing circuitry 150 causes the generation function 136 to generate a medical image related to the subject P based on the optical image and/or the skeleton information. Examples of the medical image include an image for positioning for main imaging on the subject P, an image for discrimination of at least one of an organ and a skeleton in the subject P, and an image for diagnostic imaging on the subject P. The image for positioning is, for example, an image corresponding to a positioning image generated by preliminary imaging (prescan). The image for discrimination of at least one of an organ and a skeleton corresponds to, for example, a segmentation image obtained by applying segmentation processing to a medical image. The image for diagnostic imaging corresponds to, for example, an image generated by main imaging (main scan).

The medical image is generated using, for example, a learned model. In this case, the generation function 136 reads out the learned model stored in the storage circuitry 132 from the storage circuitry 132 prior to the generation of the medical image. The learned model is learned in advance to output a medical image corresponding to an optical image by inputting the optical image. A generative artificial intelligence (AI) corresponding to the learned model is generated by training various neural networks such as generative adversarial networks (GANs), a diffusion model, or Unet by a known training method. The learned model that outputs a medical image is hereinafter referred to as an image output learned model.

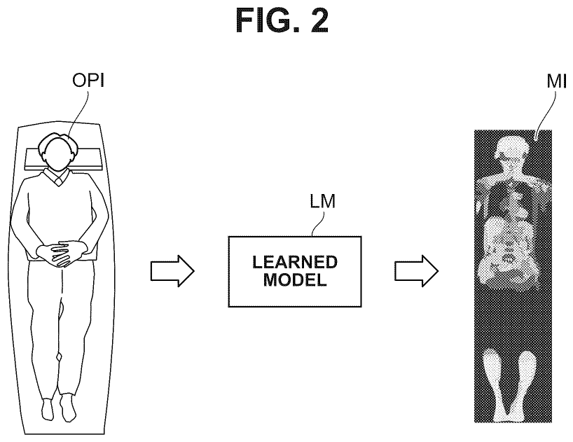

FIG. 2 illustrates an example of generation of a medical image. As illustrated in FIG. 2, the generation function 136 inputs an optical image OPI to an image output learned model LM, thereby generating a medical image MI related to the subject P. The medical image MI is a pseudo preliminarily-captured image corresponding to a preliminarily-captured image obtained by preliminary imaging (prescan) executed prior to main imaging (main scan) on the subject P. That is, according to the present embodiment, the medical image MI corresponding to the preliminarily-captured image can be generated without executing preliminary imaging on the subject P. In other words, the generation of the medical image MI corresponding to the preliminarily-captured image without executing preliminary imaging eliminates the need for preliminary imaging.

The medical image MI generated by the generation function 136 based on the optical image OPI is a positioning image to be used for, for example, positioning of the subject P in main imaging and/or setting of a posture of the subject P in main imaging. The medical image MI generated by the generation function 136 based on the optical image OPI is an image that can be visually recognized as a main captured image obtained by main imaging or a preliminarily-captured image obtained by preliminary imaging. In other words, the medical image (pseudo preliminarily-captured image) MI is an image that looks like the preliminarily-captured image. In other words, the medical image MI is an image that can be used in place of the preliminarily-captured image.

The image output learned model that outputs a medical image related to the subject P based on an input of an optical image is generated by, for example, training a GAN or a diffusion model using the optical image as training data and using the medical image corresponding to the optical image and/or skeleton information as ground truth data. The ground truth data indicates, for example, a main captured image or a preliminarily-captured image that is obtained by capturing an image of a subject P that is different from the subject P.

The optical image input to the image output learned model is not limited to the image acquired by the optical imaging device 10, but instead may be skeleton information generated by a known method based on the optical image OPI acquired from the optical imaging device 10. In other words, the generation function 136 inputs an optical image and/or skeleton information to the image output learned model, thereby generating the medical image MI. The skeleton information is, for example, data (parameter) indicating a skeleton image generated by segmentation processing or the like on the optical image OPI, or the layout (positional relationship) of the skeleton of the subject P.

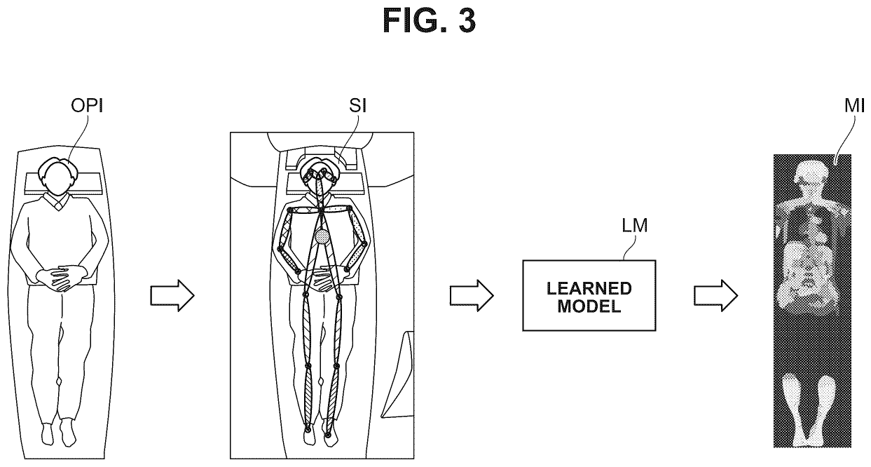

FIG. 3 illustrates an example of generation of the medical image MI using skeleton information SI. As illustrated in FIG. 3, the generation function 136 generates the medical image (pseudo preliminarily-captured image) MI by inputting the skeleton information SI generated based on the optical image OPI to the image output learned model LM. In this case, the image output learned model LM is generated by, for example, training a GAN or a diffusion model using skeleton information as training data and using the medical image corresponding to the skeleton information as ground truth data.

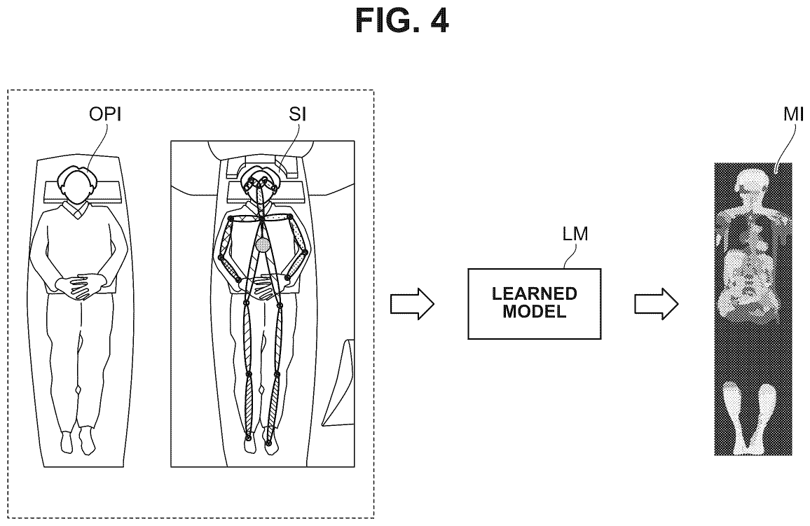

Both of the optical image OPI and the skeleton information SI may be input to the image output learned model. FIG. 4 illustrates an example of generation of the medical image MI using the skeleton information SI and the optical image OPI. As illustrated in FIG. 4, the generation function 136 generates the medical image MI by inputting the optical image OPI and the skeleton information SI to the image output learned model LM. In this case, the image output learned model is generated by, for example, training a GAN or a diffusion model using skeleton information and an optical image as training data and using the medical image corresponding to the optical image as ground truth data.

For example, imaging conditions such as a breathing condition may be further input to the image output learned model. Examples of the breathing condition include natural breathing, inspiration breath-hold if the chest of the subject P is to be imaged, and expiration breath-hold if the abdominal area of the subject P is to be imaged. The imaging conditions are not limited to the breathing condition, but also include, for example, an end-diastole on an electrocardiographic waveform.

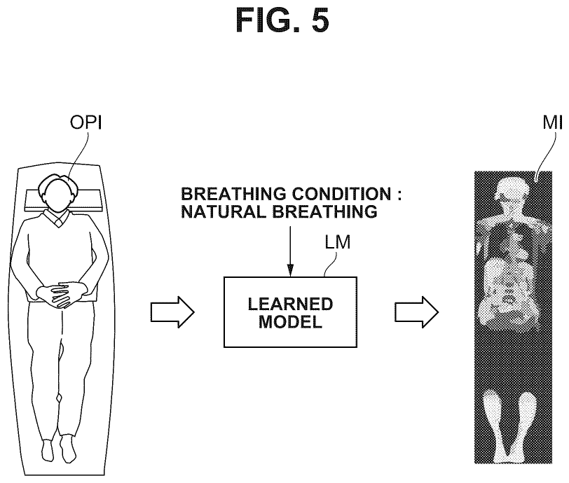

FIG. 5 illustrates an example of generation of the medical image MI by further using the breathing condition. In this case, the generation function 136 extracts the breathing condition using known character recognition processing or the like, for example, from an examination order for the subject P. For example, the examination order is transmitted from radiology information systems (RIS) to the MRI apparatus 100 and is stored in the storage circuitry 132.

In the examination order, various types of conditions (a breathing condition, imaging conditions, a posture condition indicating the posture of the subject P in an examination, and a region to be imaged in main imaging (hereinafter referred to as an imaging region) etc.) for the examination on the subject P are described. After the breathing condition is extracted, the generation function 136 inputs the optical image OPI and/or the skeleton information and the breathing condition (natural breathing) to the image output learned model LM, thereby generating the medical image MI as illustrated in FIG. 5. In this case, the image output learned model LM is generated by, for example, training a GAN or a diffusion model by further using imaging conditions as training data.

The medical image MI may be an image that can be visually recognized as with a segmentation image extracted from a preliminarily-captured image obtained by preliminary imaging executed prior to main imaging on the subject P or a main captured image obtained by main imaging. The segmentation image is, for example, an image generated by extracting an area corresponding to a predetermined region from the positioning image. Examples of the predetermined region include a bone, an organ, and a centerline of a vertebral body. In other words, the medical image MI may be an image that looks similar to the segmentation image. The generation function 136 may generate the segmentation image by known segmentation processing based on the medical image (pseudo preliminarily-captured image). In this case, the generation function 136 causes the storage circuitry 132 to store the generated segmentation image.

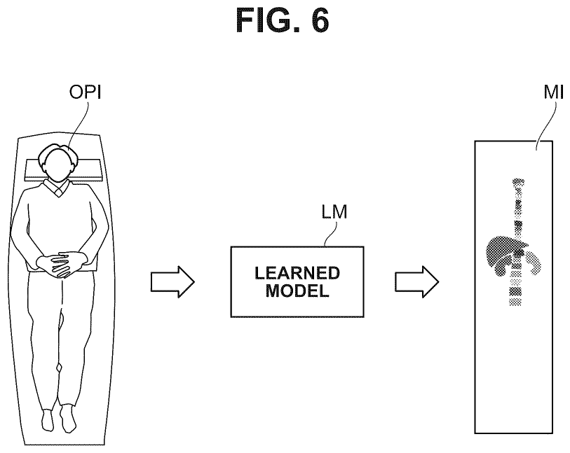

FIG. 6 illustrates an example of generation of the medical image MI corresponding to the segmentation image. As illustrated in FIG. 6, the generation function 136 generates the segmentation image MI by inputting the optical image OPI and/or the skeleton information SI to the image output learned model LM. In this case, the image output learned model LM is generated by, for example, training Unet by using an optical image as training data, using the segmentation image corresponding to the optical image as ground truth data, and using a binary cross-entropy loss, a dice loss, or the like as a loss function.

The medical image MI generated by the generation function 136 based on the optical image OPI may correspond to a medical image that can be generated by a single medical image diagnostic apparatus, or may correspond to a fusion image obtained by combining a plurality of medical images that can be generated by a plurality of medical image diagnostic apparatuses. In these cases, the image output learned model LM is generated by, for example, training a GAN or a diffusion model by using an optical image as training data and using a medical image or a fusion image that can be generated by a single medical image diagnostic apparatus corresponding to the optical image as ground truth data.

For example, like in the present embodiment, if the MRI apparatus 100 is used as the medical image diagnostic apparatus, a single medical image diagnostic apparatus corresponds to the MRI apparatus 100. In this case, the medical image MI generated by the generation function 136 based on the optical image OPI corresponds to an MR image. The medical image MI generated based on the optical image OPI is not limited to an MR image and depends on learning data used to generate the image output learned model LM. Specifically, if a CT image is used as ground truth data, the medical image MI to be generated based on the optical image OPI is a pseudo CT image. If a fusion image obtained by combining a CT image with an MR image is used as ground truth data, the medical image MI to be generated based on the optical image OPI is a pseudo MRI-CT image obtained by fusing a pseudo MR image with a pseudo CT image.

If a PET-MRI apparatus is used as the medical image diagnostic apparatus, the medical image MI generated by the generation function 136 based on the optical image OPI corresponds to a fusion image obtained by fusing a PET image with an MR image. If an SPECT-MRI apparatus is used as the medical image diagnostic apparatus, the medical image MI generated by the generation function 136 based on the optical image OPI corresponds to a fusion image obtained by fusing an SPECT image with an MR image.

For example, if an X-ray CT apparatus is used as the medical image diagnostic apparatus, a single medical image diagnostic apparatus corresponds to the X-ray CT apparatus. In this case, the medical image MI generated by the generation function 136 based on the optical image OPI corresponds to a CT image (e.g., scanogram). If a PET-CT apparatus is used as the medical image diagnostic apparatus, the medical image MI generated by the generation function 136 based on the optical image OPI corresponds to a fusion image obtained by fusing a PET image with a CT image. If an SPECT-CT apparatus is used as the medical image diagnostic apparatus, the medical image MI generated by the generation function 136 based on the optical image OPI corresponds to a fusion image obtained by fusing an SPECT image with a CT image.

The medical image MI generated by the generation function 136 based on the optical image OPI may correspond to at least one of a plurality of sections in the subject P. Examples of the plurality of sections include a sagittal section, a coronal section, and an axial section of the subject P. The plurality of sections is not limited to the above-described examples. The plurality of sections may also include an oblique section.

In these cases, the image output learned model LM is generated by, for example, training a GAN or a diffusion model by using an optical image as training data and using a medical image corresponding to at least one of a plurality of sections as ground truth data. In this case, the medical image MI generated by the generation function 136 based on the optical image OPI is an image obtained by visualizing anatomical information about the subject P. Examples of the anatomical information include various organs and bones of the subject P.

A method for generating the medical image MI by the generation function 136 using the image output learned model LM has been described above, but the medical image MI generation method is not limited to this example. For example, the generation function 136 may generate the medical image MI related to the subject P based on a previous optical image (hereinafter referred to as a previous optical image), a medical image corresponding to previous main imaging (hereinafter referred to as a previous main captured image) and a current optical image (hereinafter referred to as a current optical image).

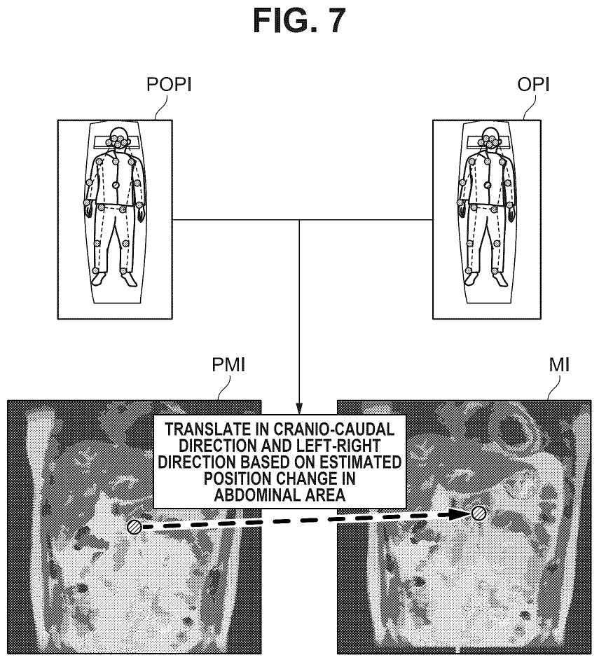

FIG. 7 illustrates an example of generation of the medical image MI based on a previous optical image POPI, a previous main captured image PMI, and the current optical image OPI. As illustrated in FIG. 7, the generation function 136 calculates a correspondence relation between the current optical image OPI and the previous optical image POPI of the subject P. As illustrated in FIG. 7, the correspondence relation indicates, for example, a change in the position of an abdominal area (anatomical landmark) of the subject P. In other words, the generation function 136 calculates a position change (estimated position change) estimated in the abdominal area of the subject P.

Next, the generation function 136 generates the medical image MI based on the calculated correspondence relation and the medical image (previous main captured image) PMI obtained by previous main imaging of the subject P. Specifically, the generation function 136 generates the medical image MI by moving the previous main captured image PMI in a direction parallel to a craniocaudal direction and a left-right direction, for example, based on the estimated position change in the abdominal area of the subject P as illustrated in FIG. 7.

The generation function 136 reads out k-space data collected by main imaging on the subject P from the storage circuitry 132. Then, the generation function 136 performs reconstruction processing such as Fourier transform on the read k-space data, thereby generating an MR image. A known method can be applied as the reconstruction processing, and thus the description thereof is omitted. The processing circuitry 150 that implements the generation function 136 is an example of a generation unit.

The processing circuitry 150 causes the determination function 138 to determine whether main imaging can be executed based on a medical image (pseudo preliminarily-captured image) MI. For example, the determination function 138 applies the medical image MI to a learned model that outputs a posture of the subject P (this learned model is hereinafter referred to as a posture output learned model) by input of a medical image MI, thereby determining the posture state of the subject P. The posture output learned model is implemented by, for example, a learned convolutional neural network (CNN). The posture output learned model is a CNN that outputs the posture of the subject P in the medical image MI based on an input of the medical image MI. Examples of the posture of the subject P include a forward-bending position, a backward-bending position, and a neutral position between the forward-bending position and the backward-bending position. The posture of the subject P may be defined by an angle of the subject P in the craniocaudal direction.

Training data used to generate the posture output learned model is generated by, for example, the following procedure. First, a segmentation image of a vertebral body (hereinafter referred to as a vertebral body image) is generated by inputting a preliminarily captured MR image to a known cycle GAN. Assuming that the size of each vertebra area in the vertebral body image is set to be constant and the area of the intervertebral disc between the adjacent vertebra areas is set to be deformable, the angle of the vertebral body is changed and the position of the vertebral body rotated about the center of the image is restored (e.g., restored by the amount of movement corresponding to centroid coordinates of each bone), thereby generating the vertebral body image corresponding to the posture of the human body (hereinafter referred to as a posture vertebral body image).

Specifically, linear transformation corresponding to the angle is executed on the vertebral body image assuming that the vertebra is a rigid body and the intervertebral disc is an elastic body. Any other transformation method may be used instead of the linear transformation. The angle (angle of vertebral body) obtained by linear transformation is set according to the posture of the human body. The angle of the vertebral body is associated with the posture of the human body. Then, the posture vertebral body image is input to a generator in the cycle GAN, to thereby generate a medical image for learning based on the posture of the human body.

The posture output learned model is generated by training a CNN to be trained using the medical image for learning and the posture of the human body associated with the medical image for learning. Specifically, the medical image for learning is input to the CNN to be trained and the CNN to be trained is trained using a backpropagation method so as to reduce errors based on the difference between an output result from the CNN and the posture of the human body (ground truth data) associated with the medical image for learning. Thus, the posture output learned model is generated.

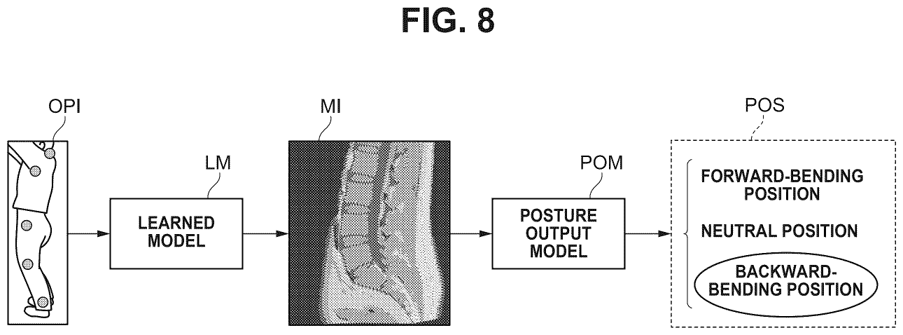

FIG. 8 illustrates an example of determination of the posture state of the subject P by applying the medical image MI to a posture output learned model POM. As illustrated in FIG. 8, the determination function 138 inputs the medical image MI generated based on the input of the optical image OPI to the image output learned model LM to the posture output learned model POM. Thus, the determination function 138 determines the posture (backward-bending position in FIG. 8) of the subject P corresponding to the optical image OPI among a plurality of posture states POS.

The determination function 138 extracts the posture of the subject P in main imaging of the subject P from the examination order. The determination function 138 compares the posture extracted from the examination order with the posture of the subject P generated by the posture output learned model POM. If the posture extracted from the examination order matches the posture of the subject P generated by the posture output learned model POM, the determination function 138 determines that main imaging on the subject P can be executed. If the posture extracted from the examination order does not match the posture of the subject P generated by the posture output learned model POM, the determination function 138 determines that main imaging on the subject P cannot be executed.

The determination function 138 extracts an imaging region from the examination order. The determination function 138 determines whether the position of a projector provided on the ceiling of the examination room in which the MRI apparatus 100 is placed includes the center of the imaging region. Specifically, the determination function 138 determines whether the projector position matches a predetermined area (hereinafter referred to as a central portion) including the center of the imaging region.

The determination as to whether to execute main imaging is not limited to the automatic determination as described above. For example, the determination may be made according to a user instruction (e.g., operation of an operator indicating whether main imaging can be executed, a button, and the like) via the input device 141. The processing circuitry 150 that implements the determination function 138 is an example of a determination unit.

The processing circuitry 150 causes the display control function 140 to display a setting screen for setting imaging conditions, a scan plan, and the like for the subject P on the display 143. The display control function 140 presents the medical image generated by the generation function 136. In other words, the display control function 140 causes the display 143 to display the pseudo preliminarily-captured image. Further, the display control function 140 causes the display 143 to display the MR image generated by the generation function 136.

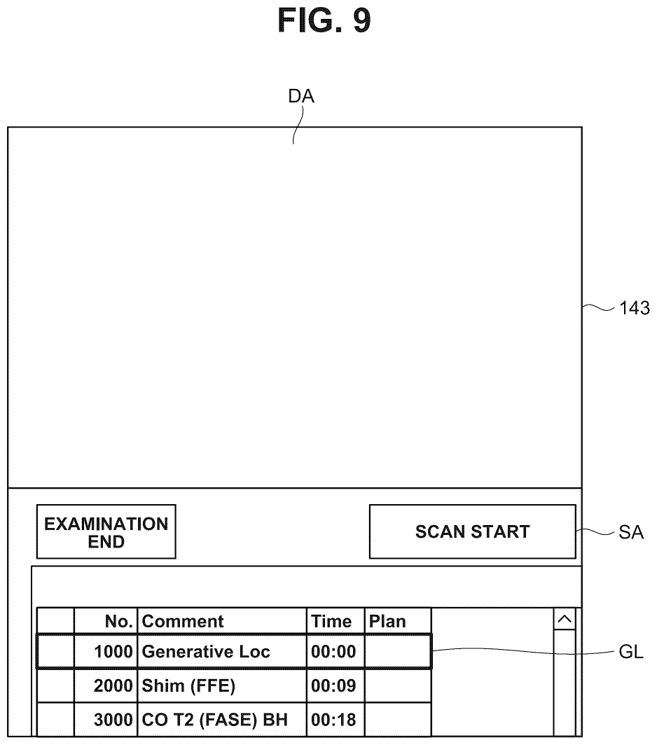

FIG. 9 illustrates an example of a display screen to select imaging (scan) for the subject P. As illustrated in FIG. 9, in the present embodiment, a field for generating the medical image MI instead of positioning scan (locator scan) (this field is hereinafter referred to as medical image generation field) Generative Locator (Loc) (GL) is displayed. FIG. 9 illustrates a state where the medical image generation field GL No. 1000 is selected by the user. In this case, when a scan start button SA is operated (e.g., double-clicked), optical imaging is executed on the subject P, and the display control function 140 causes the display 143 to display the generated medical image MI on an image display field DA.

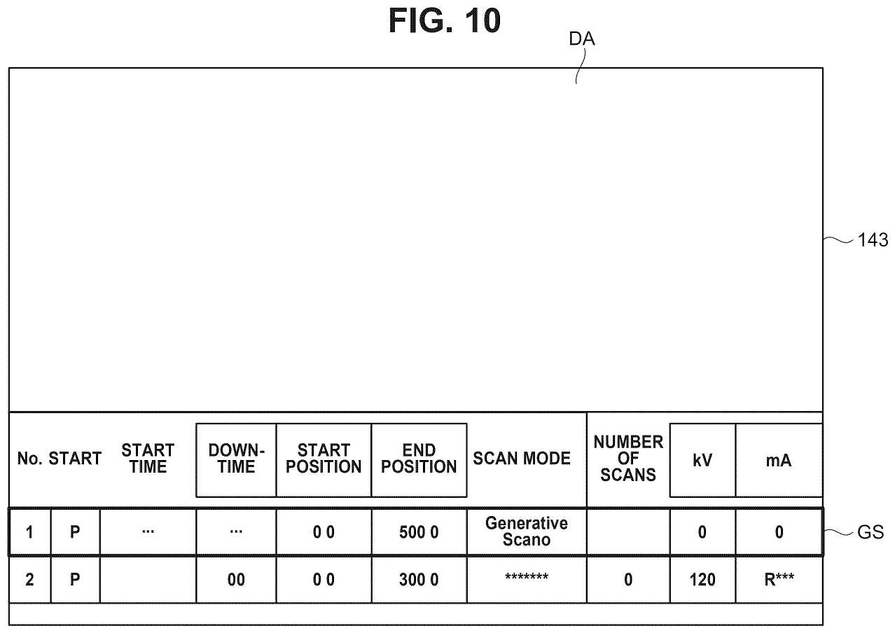

FIG. 10 illustrates an example of a display screen to select imaging (scan) for the subject P when an X-ray CT apparatus is used as the medical image diagnostic apparatus. As illustrated in FIG. 10, in the present embodiment, a medical image generation field GS is displayed instead of scanography (Scano). FIG. 10 illustrates a state where the medical image generation field GL No. 1 is selected by the user.

As illustrated in FIG. 10, according to the present embodiment, the medical image (scanogram) MI is generated without exposing the subject P to X-rays. Accordingly, “0” is displayed as the value of each of a tube voltage (kV) and a tube current (mA). In this case, if the scan start button SA, which is not illustrated in FIG. 10, is operated, optical imaging is executed on the subject P and the display control function 140 causes the display 143 to display the generated medical image MI on the image display field DA.

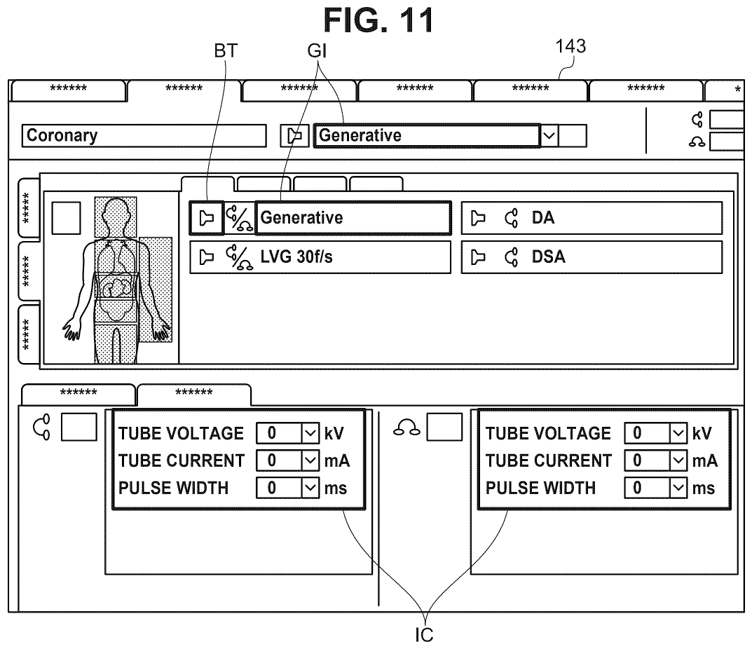

FIG. 11 illustrates an example of a display screen to select imaging for the subject P when an X-ray diagnostic apparatus is used as the medical image diagnostic apparatus. As illustrated in FIG. 11, in the present embodiment, a medical image generation field GI is displayed instead of preliminary imaging. FIG. 11 illustrates a state where the medical image generation field GI is selected by the user.

As illustrated in FIG. 11, according to the present embodiment, the medical image MI is generated without exposing the subject P to X-rays. Accordingly, “0” is displayed as the value of each of the tube voltage (kV), the tube current (mA), and a pulse width in imaging conditions IC. In this case, when an ON button BT illustrated in FIG. 11 is operated, optical imaging is executed on the subject P and the display control function 140 causes the display 143 to display the generated medical image MI on the image display field DA (not illustrated).

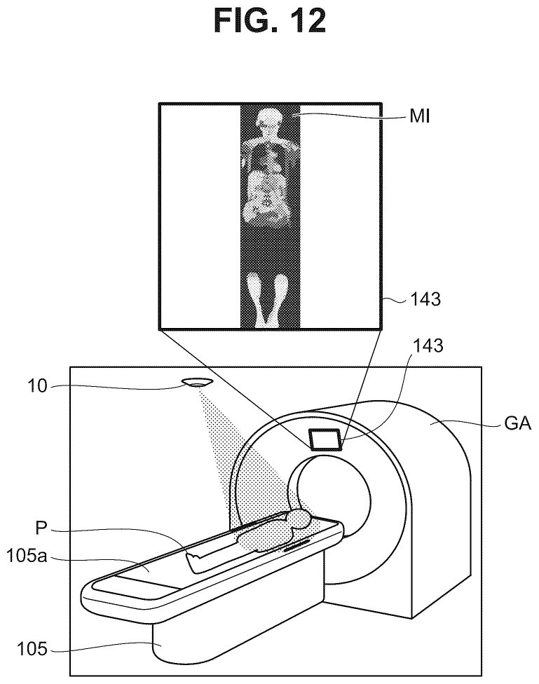

While FIGS. 9 to 11 each illustrate an example where the medical image MI is displayed on the display 143 in the computer 130 (or console), the display 143 on which the medical image MI is displayed is not limited to that of the computer 130 (or console). The medical image MI may be displayed on the display 143 provided on a gantry GA in the MRI apparatus 100, the X-ray CT apparatus, or the like as illustrated in FIG. 12.

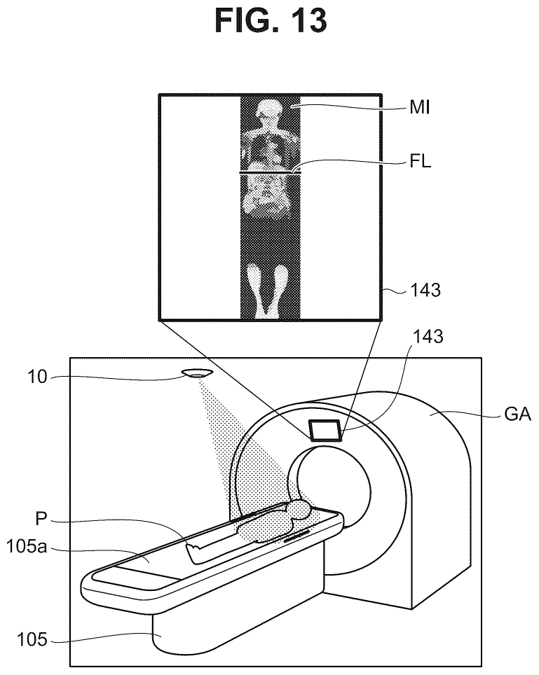

The display control function 140 may cause the display 143 to display the imaging range for main imaging to be implemented by the setting function 142 to be described below, the projector position, and the like by superimposing the imaging range, the projector position, and the like on the medical image MI. A display example for setting the imaging range will be described below. FIG. 13 illustrates an example of a projector position FL superimposed on the medical image MI. The projector position FL in the medical image MI is calculated by a predetermined calculation based on, for example, a relative positional relationship between the position of the optical imaging device 10 and the projector position FL and the imaging range of the optical imaging device 10. As illustrated in FIG. 13, the user can easily recognize the relative positional relationship between the medical image MI and the projector.

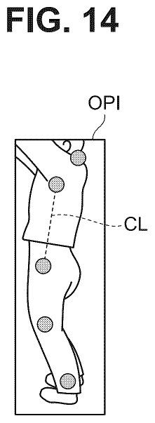

For example, the display control function 140 may cause the display 143 provided on the gantry GA to display the segmentation image (segmentation result) generated by the generation function 136 by superimposing the segmentation image on the optical image OPI. In this case, the display 143 displays a posture state by superimposing the posture state on the optical image OPI. For example, as illustrated in FIG. 14, the display control function 140 causes the display 143 provided on the gantry GA to display a centerline CL of the vertebral body as a result of segmentation by superimposing the centerline CL on the optical image OPI. The processing circuitry 150 that implements the display control function 140 is an example of a display control unit.

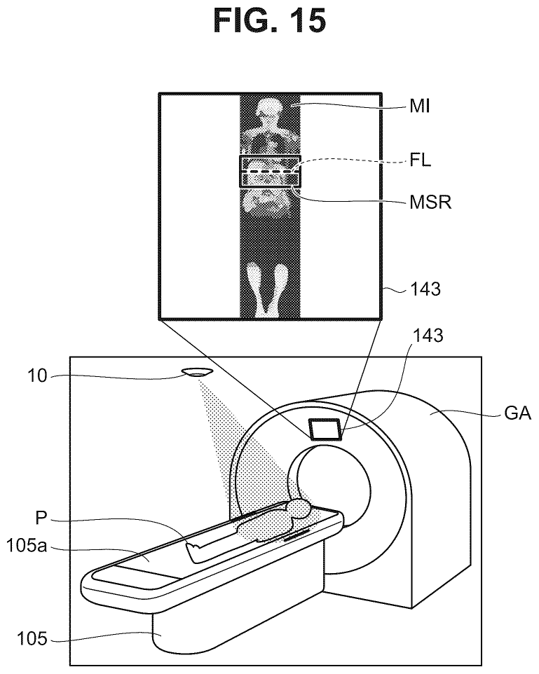

The processing circuitry 150 causes the setting function 142 to set an imaging range for main imaging of the subject P (this imaging range is hereinafter referred to as a main imaging range) based on the projector position FL in the medical image (pseudo preliminarily-captured image) MI. Specifically, if positioning for main imaging on the subject P is determined (hereinafter referred to as positioning determination), the setting function 142 sets a predetermined range around the projector position FL in the medical image MI as the main imaging range. In this case, as illustrated in FIG. 15, the display control function 140 causes the display 143 to display the projector position FL and a main imaging range MSR by superimposing the projector position FL and the main imaging range MSR on the medical image MI. The processing circuitry 150 that implements the setting function 142 is an example of a setting unit.

If long length imaging is designated as main imaging in the examination order for the subject P, the setting function 142 sets a plurality of main imaging ranges corresponding to long length imaging on the medical image MI based on the projector position FL when positioning is determined. Examples of an imaging target in long length imaging include the entire spine, whole body, and lower limb of the subject P. According to the setting of the plurality of main imaging ranges, the display control function 140 causes the display 143 to display the plurality of main imaging ranges corresponding to long length imaging by superimposing the plurality of main imaging ranges together with the projector position FL on the medical image MI.

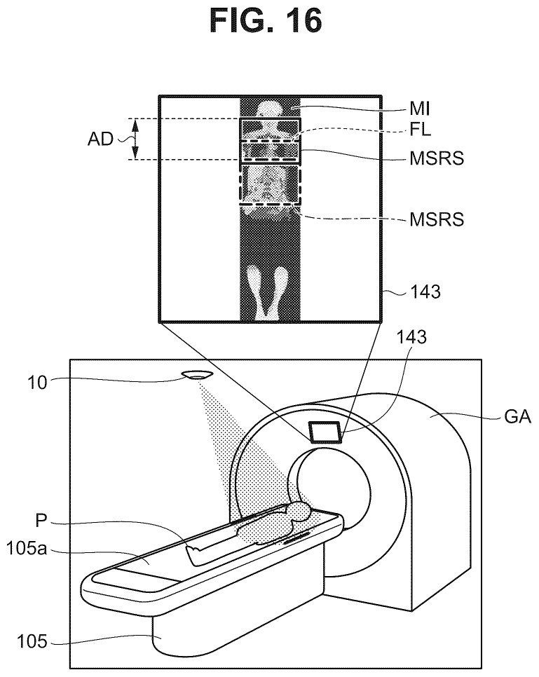

For example, if the entire spine is set as the imaging target in long length imaging, as illustrated in FIG. 16, the display control function 140 causes the display 143 to display a plurality of main imaging ranges MSRS corresponding to long length imaging by superimposing the plurality of main imaging ranges MSRS together with the projector position FL on the medical image MI. A positional relationship between two adjacent main imaging ranges among the plurality of main imaging ranges MSRS, or a deviation amount AD between two main imaging ranges is preliminarily set through designation by the user via the input device 141 during setting of imaging conditions, and is stored in the storage circuitry 132.

If positioning for main imaging on the subject P is not determined, or if positioning for main imaging on the main imaging range MSR illustrated in FIG. 15 or the plurality of main imaging ranges MSRS illustrated in FIG. 16 is not determined by the user, for example, the setting function 142 adjusts and resets the projector position FL according to a user instruction via the input device 141.

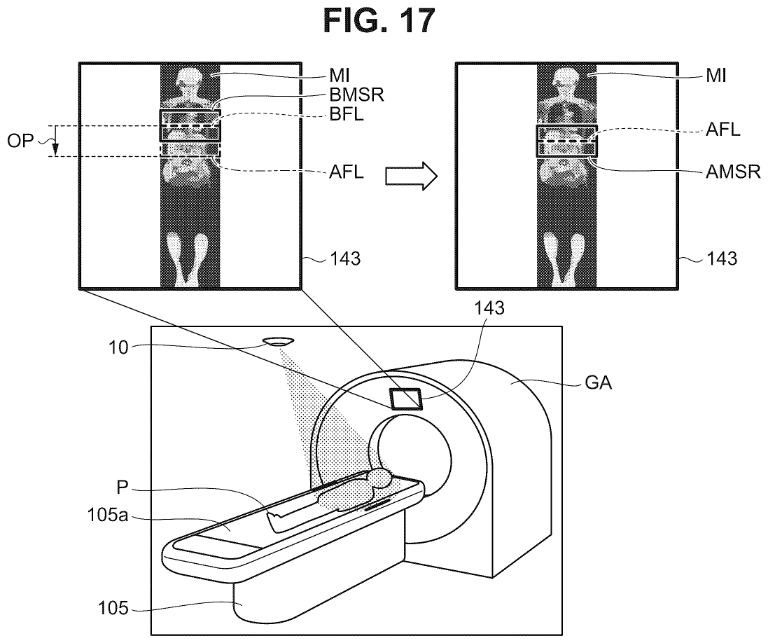

FIG. 17 illustrates an example of adjustment of the imaging range MSR. As illustrated in FIG. 17, a projector position BFL before resetting is different from a projector position desired by the user (this projector position is hereinafter referred to as a desired position) AFL. The position BFL before resetting is moved to the desired position AFL in the medical image MI representing anatomical information about the subject P on a sagittal section in a pseudo manner according to a user instruction via the input device 141. The couch control circuitry 106 moves the couch 105 or the couchtop 105a in a direction opposite to a movement amount OP according to the position BFL before resetting and the movement amount OP.

Thus, as illustrated in FIG. 17, the projector position BFL before resetting is moved to the desired position AFL representing the reset projector position desired by the user. Then, as illustrated in FIG. 17, the setting function 142 resets a main imaging range BMSR before resetting to a reset imaging range AMSR desired by the user.

The setting function 142 may set the projector position FL based on the current projector position and an imaging target region in the segmentation image as the medical image MI. Specifically, if the projector position does not match the central portion of the imaging region, the setting function 142 sets the amount of movement of the couch 105 or the couchtop 105a so that the projector position can match the central portion of the imaging region. The couch control circuitry 106 moves the couch 105 or the couchtop 105a by the set amount of movement. Thus, the projector position in the segmentation image is set to be located in the central portion of the imaging region.

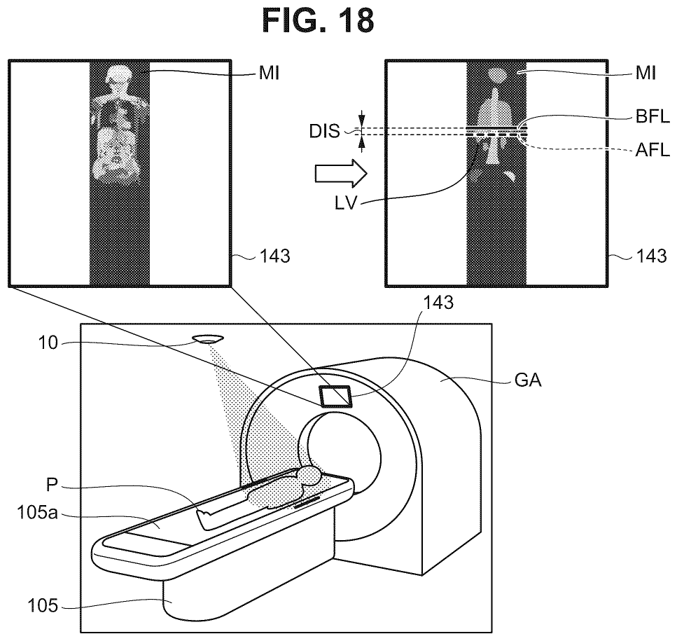

FIG. 18 illustrates an example of automatically moving the projector position. As illustrated in FIG. 18, a liver LV is set as an imaging region. As illustrated in FIG. 18, the determination function 138 executes segmentation processing on the pseudo preliminarily-captured image MI displayed on the display 143. Thus, the segmentation image corresponding to the pseudo preliminarily-captured image MI is generated as the medical image MI. The segmentation processing on the pseudo preliminarily-captured image MI may be implemented by the generation function 136.

Next, the determination function 138 determines whether the projector position BFL before resetting in the segmentation image MI matches the central portion of the imaging region (liver LV). In the example illustrated in FIG. 18, a central portion of the imaging region (liver LV) is spaced apart from the projector position BFL in the segmentation image MI by a distance DIS. In this case, the setting function 142 sets the central portion of the imaging region (liver LV) as a new projector position AFL.

Then, the setting function 142 calculates the distance DIS illustrated in FIG. 18 using the new projector position AFL and the projector position BFL before resetting. The couch control circuitry 106 moves the couch 105 or the couchtop 105a by the set amount of movement. Thus, the setting function 142 automatically sets the projector position AFL in the central portion of the imaging region (liver LV) as illustrated in FIG. 18.

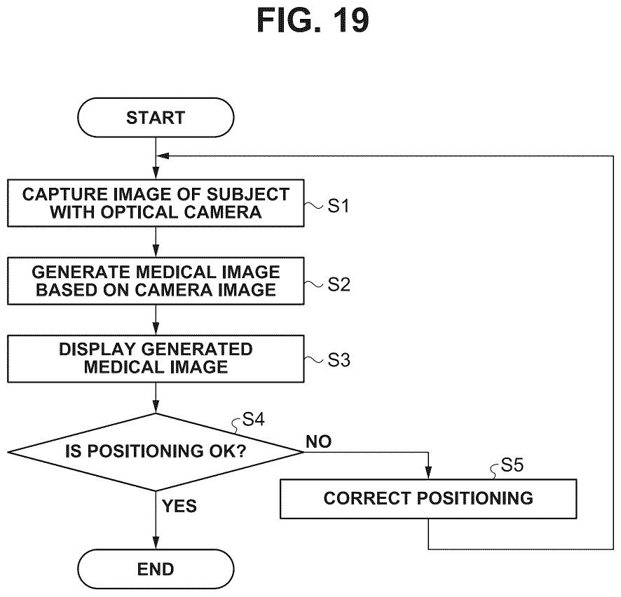

The overall configuration of the MRI apparatus 100 according to the embodiment has been described above. In this configuration, the MRI apparatus 100 according to the embodiment generates the medical image MI (pseudo preliminarily-captured image corresponding to the preliminarily-captured image) without performing preliminary imaging (locator imaging), and executes processing of setting an imaging range for main imaging (main scan) on the subject P (this processing is hereinafter referred to as non-imaging positioning processing). A procedure for non-imaging positioning processing will be described below with reference to FIG. 19. FIG. 19 is a flowchart illustrating an example of a non-imaging positioning processing procedure.

### Non-Imaging Positioning Processing

### Step S1

The input device 141 inputs an instruction to capture an optical image of the subject P. For example, if the input device 141 is mounted on the gantry GA, the user operates an operator displayed on the display 143 of the gantry GA or a switch provided in the vicinity of the display 143, thereby inputting the instruction to capture an optical image of the subject P to the input device 141.

The optical imaging device 10 captures an image of the subject P according to the optical imaging instruction. Thus, the optical imaging device 10 generates the optical image OPI including the subject P placed on the couchtop 105a. The optical imaging device 10 outputs the generated optical image OPI to the MRI apparatus 100. The processing circuitry 150 causes the acquisition function 134 to acquire the optical image OPI. The acquisition function 134 causes the storage circuitry 132 to store the acquired optical image OPI.

### Step S2

The processing circuitry 150 causes the generation function 136 to generate the medical image MI based on the camera image (optical image OPI). Specifically, the generation function 136 generates the medical image MI as the pseudo preliminarily-captured image corresponding to the preliminarily-captured image (locator image) based on the optical image OPI. For example, the generation function 136 inputs the optical image OPI to the image output learned model LM. The generation function 136 generates the medical image MI based on an output from the image output learned model LM. The method for generating the medical image MI is not limited to the method using the image output learned model LM. As illustrated in FIG. 7, the medical image MI may be generated based on the previous optical image POPI, the previous main captured image PMI, and the current optical image OPI. The generation function 136 causes the storage circuitry 132 to store the generated medical image MI.

### Step S3

The processing circuitry 150 causes the display control function 140 to display the generated medical image MI on the display 143. As a result, the display 143 displays the medical image MI, for example, as illustrated in FIG. 12. Further, as illustrated in FIG. 13, the display control function 140 may cause the display 143 to display the projector position FL by superimposing the projector position FL on the medical image MI. For example, as illustrated in FIG. 15, the display control function 140 may cause the display 143 to display not only the projector position FL but also the imaging range MSR by superimposing the projector position FL and the main imaging range MSR on the medical image MI.

If the entire spine is set as the imaging target in long length imaging on the subject P, the display control function 140 may cause the display 143 to display the plurality of main imaging ranges MSRS together with the projector position FL by superimposing the plurality of main imaging ranges MSRS and the projector position FL on the medical image MI, for example, as illustrated in FIG. 16. Further, the display control function 140 may cause the display 143 to display the centerline CL of the body as a segmentation result by superimposing the centerline CL on the medical image MI, for example, as illustrated in FIG. 14.

### Step S4

The processing circuitry 150 causes the determination function 138 to determine whether main imaging can be executed based on the medical image MI. The determination as to whether main imaging can be executed corresponds to a determination as to whether positioning is OK. For example, information indicating whether positioning is OK is input as a user instruction via the input device 141. If positioning is OK (YES in step S4), the non-imaging positioning processing ends. In this case, main imaging is executed on the subject P. If positioning is not OK (NO in step S4), the processing proceeds to step S5.

The determination as to whether main imaging can be executed is not limited to a determination based on a user instruction via the input device 141, but instead may be automatically executed by the MRI apparatus 100. For example, the determination function 138 inputs the medical image MI to the posture output learned model POM and outputs the posture of the subject P in the medical image MI. The determination function 138 compares the posture of the subject P output from the posture output learned model POM with the posture of the subject P for main imaging described in the examination order.

The generation function 136 executes segmentation processing on the medical image MI including anatomical information about the subject P. Next, the determination function 138 determines whether the projector position FL in the segmentation image is located in the central portion of the imaging region described in the examination order. If the posture of the subject P output from the posture output learned model POM is identical to the posture of the subject P for main imaging described in the examination order and the projector position FL in the segmentation image is located in the central portion of the imaging region described in the examination order, the determination function 138 determines that positioning is OK (YES in step S4).

If the posture of the subject P output from the posture output learned model POM is different from the posture of the subject P described in the examination order, or if the projector position FL in the segmentation image is not located in the central portion of the imaging region described in the examination order, the determination function 138 determines that positioning is not OK (NO in step S4).

### Step S5

The processing circuitry 150 causes the setting function 142 to adjust and reset the projector position FL according to a user instruction via the input device 141. Thus, positioning in main imaging is corrected. For example, as illustrated in FIG. 17, when the projector position BFL is moved to the desired position AFL according to a user instruction via the input device 141, the couch control circuitry 106 moves the couch 105 or the couchtop 105a in a direction opposite to the movement amount OP.

As illustrated in FIG. 18, the couch control circuitry 106 moves the couch 105 or the couchtop 105a so that the projector position AFL is located in the central portion of the imaging region (liver LV) in the segmentation image MI. Accordingly, the setting function 142 automatically sets the projector position AFL in the central portion of the imaging region (liver LV) as illustrated in FIG. 18. After step S5, the processing of step S1 and subsequent steps is repeated.

When the non-imaging positioning processing ends (YES in step S4), the input device 141 sets the main imaging range MSR through a user operation. When the main imaging range MSR is set, the input device 141 inputs an instruction to start main imaging (main scan) according to a user operation. The sequence control circuitry 120 executes main imaging (main scan) on the set main imaging range MSR in response to the main imaging start instruction.

The sequence control circuitry 120 may execute main imaging when positioning is determined. When main imaging is executed, the acquisition function 134 acquires k-space data and causes the storage circuitry 132 to store the k-space data. The generation function 136 executes reconstruction processing on the k-space data to generate an MR image corresponding to main scan. The display control function 140 causes the display 143 to display the generated MR image.

If the non-imaging positioning processing is executed in the medical image diagnostic apparatus, after positioning determination, the setting function 142 sets an imaging range (main imaging range) for main imaging on the subject P in the pseudo preliminarily-captured image MI. Next, the imaging unit executes main imaging on the set imaging range. The imaging unit may be referred to as an imaging apparatus. If the medical image diagnostic apparatus is implemented as an X-ray CT apparatus, a PET apparatus, an SPECT apparatus, a PET-CT apparatus, an SPECT-CT apparatus, a PET-MRI apparatus, or an SPECT-MR apparatus, the imaging unit corresponds to, for example, a gantry in these apparatuses. If the medical image diagnostic apparatus is implemented as an X-ray diagnostic apparatus, the imaging unit corresponds to various types of devices (X-ray tube, X-ray detector, etc.) mounted on, for example, a C-arm and/or an Ω-arm in the X-ray diagnostic apparatus.

The MRI apparatus 100 according to the embodiment described above acquires the optical image OPI of the subject obtained by imaging using the optical imaging device 10 and/or the skeleton information SI about the subject P, generates the medical image MI related to the subject P based on the optical image OPI and/or the skeleton information SI, and presents the medical image MI. Further, the MRI apparatus 100 according to the embodiment calculates the correspondence relation between the optical image OPI and the previous optical image of the subject P, and generates a medical image related to the subject P based on the previous medical image obtained by main imaging of the subject P and the correspondence relation.

The medical image MI in the MRI apparatus 100 according to the embodiment corresponds to the pseudo preliminarily-captured image corresponding to the preliminarily-captured image obtained by preliminary imaging executed prior to main imaging on the subject P. The medical image MI in the MRI apparatus 100 according to the embodiment is an image that can be visually recognized in a similar manner as the preliminarily-captured image, an image that can be visually recognized in a similar manner as the segmentation image extracted from the preliminarily-captured image, a medical image that can be generated by a single medical image diagnostic apparatus, a fusion image obtained by combining a plurality of medical images that can be generated by a plurality of medical image diagnostic apparatuses, at least one of a plurality of sections in the subject P, or an image obtained by visualizing anatomical information about the subject P.

The MRI apparatus 100 according to the embodiment sets the imaging range MSR for main imaging of the subject P in the pseudo preliminarily-captured image MI, and executes main imaging on the imaging range MSR. In this case, the MRI apparatus 100 according to the embodiment may display the imaging range in main imaging by superimposing the imaging range on the medical image MI. The above-described configurations enable the MRI apparatus 100 according to the embodiment to execute main imaging by setting the imaging range MSR for main imaging without performing preliminary imaging before main imaging.

In view of the above, the MRI apparatus 100 according to the embodiment can generate the medical image MI used for positioning for main imaging without performing preliminary imaging (positioning imaging (prescan)) on the subject P. With this configuration, the MRI apparatus 100 according to the embodiment can shorten the time required for an MR examination on the subject P, which leads to an improvement in throughput of an MR examination. In addition, the MRI apparatus 100 according to the embodiment can reduce the burden of an MR examination on the subject P. Further, if the medical image diagnostic apparatus is implemented as any one of an X-ray CT apparatus, an X-ray diagnostic apparatus, a PET apparatus, an SPECT apparatus, a PET-CT apparatus, and an SPECT-CT apparatus, the exposure of the subject P to X-rays can be reduced in addition to the above-described advantageous effects.

Furthermore, the MRI apparatus 100 according to the embodiment determines whether main imaging can be executed based on the medical image (preliminarily-captured image) MI. For example, the MRI apparatus 100 according to the embodiment determines the posture state of the subject P by applying the medical image MI to the posture output learned model POM that outputs the posture of the subject P based on an input of the medical image MI. The posture state in the MRI apparatus 100 according to the embodiment indicates a body position of the subject P and/or an angle of the subject P in the craniocaudal direction. The MRI apparatus 100 according to the embodiment displays the posture state by superimposing the posture state on the optical image OPI.

In view of the above, the MRI apparatus 100 according to the embodiment can acquire the posture state of the subject P without the need for positioning imaging, and can present the posture state to the user. Therefore, the MRI apparatus 100 according to the embodiment can guide the posture of the subject P using the posture state. In addition, the MRI apparatus 100 according to the embodiment can determine whether main imaging can be executed based on the medical image MI generated without positioning imaging. For example, the MRI apparatus 100 according to the embodiment can compare the posture of the subject P output from the posture output learned model POM with the posture of the subject P for main imaging described in the examination order, and can determine whether main imaging can be executed based on whether the projector position FL in the segmentation image MI is located in the central portion of the imaging region described in the examination order.

Consequently, the MRI apparatus 100 according to the embodiment can implement the posture of the subject P described in the examination order without performing positioning imaging, and can execute main imaging by appropriately setting the main imaging range MSR.

As described above, the MRI apparatus 100 according to the embodiment can provide various types of user interfaces such as settings of the posture of the subject P and the imaging range without positioning imaging. Therefore, the MRI apparatus 100 according to the embodiment can generate the medical image MI based on the camera image OPI and can provide various types of user interfaces without positioning imaging, which leads to an improvement in the efficiency of various types of workflow such as an MR examination.

### Modified Examples

A modified example of the embodiment uses a positioning image (preliminarily-captured image) obtained by executing preliminary imaging (prescan) as the medical image MI described in the embodiment. In other words, this modified example eliminates the need for the optical imaging device 10. This modified example also eliminates the need for generating the pseudo preliminarily-captured image by the generation function 136. In this modified example, steps S1 and S2 can be omitted in the flowchart illustrated in FIG. 19.

In this modified example, prior to step S3, the sequence control circuitry 120 executes preliminary imaging (also referred to as positioning imaging, locator imaging, or prescan) on the subject P. In this case, the generation function 136 performs reconstruction processing such as Fourier transform on k-space data collected by preliminary imaging, thereby generating the positioning image.

A technical feature according to this modified example is determining whether main imaging can be executed based on the preliminarily-captured image (positioning image). The content of processing for determining whether positioning for main imaging can be performed using the positioning image corresponds to the processing in which the medical image MI according to the embodiment is replaced with the positioning image, and thus the description thereof is omitted. The MRI apparatus 100 according to this modified example can implement the posture of the subject P described in the examination order using the positioning image and can execute main imaging by appropriately setting the main imaging range MSR. With this configuration, The MRI apparatus 100 according to this modified example can improve the efficiency of various types of workflow such as positioning of the subject P before main imaging and setting of the main imaging range.

To implement the technical idea according to the embodiments by the medical image processing method, the medical image processing method includes acquiring the optical image OPI of the subject P captured by imaging using the optical imaging device 10 and/or the skeleton information SI about the subject P, generating the medical image MI related to the subject P based on the optical image OPI and/or the skeleton information SI, and presenting the medical image MI. A procedure for non-imaging positioning processing to be executed by the medical image processing method and advantageous effects obtained by the medical image processing method are similar to those described in the embodiments, and thus descriptions thereof are omitted.

According to the embodiments and modified examples described above, it is possible to improve the efficiency of various types of examination workflow in a medical image diagnostic apparatus.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnostic apparatus comprising processing circuitry configured to:
acquire an optical image of a subject captured by an optical imaging device and/or skeleton information about the subject;
generate a medical image related to the subject based on the optical image and/or the skeleton information; and
cause a display to display the medical image.

2. The medical image diagnostic apparatus according to claim 1, wherein the medical image is an image for positioning for main imaging on the subject.

3. The medical image diagnostic apparatus according to claim 2, wherein the medical image is a pseudo preliminarily-captured image corresponding to a preliminarily-captured image obtained by preliminary imaging executed prior to the main imaging.

4. The medical image diagnostic apparatus according to claim 3, wherein the pseudo preliminarily-captured image is an image that is visually recognizable in a similar manner as the preliminarily-captured image.

5. The medical image diagnostic apparatus according to claim 3, wherein the processing circuitry is configured to determine whether the main imaging is to be executed based on the pseudo preliminarily-captured image.

6. The medical image diagnostic apparatus according to claim 3,
wherein the processing circuitry sets an imaging range for the main imaging of the subject in the pseudo preliminarily-captured image, and
wherein the processing circuitry is further configured to execute the main imaging on the imaging range.

7. The medical image diagnostic apparatus according to claim 3, wherein the processing circuitry generates the medical image by inputting the optical image and/or the skeleton information to an image output learned model trained to output the medical image by input of the optical image and/or the skeleton information.

8. The medical image diagnostic apparatus according to claim 7, wherein the processing circuitry generates the medical image by further inputting an imaging condition for the subject related to the main imaging to the image output learned model.

9. The medical image diagnostic apparatus according to claim 1, wherein the medical image is an image for discrimination of at least one of an organ and a skeleton in the subject.

10. The medical image diagnostic apparatus according to claim 9, wherein the medical image is an image visually recognizable in a similar manner as a segmentation image extracted from one of a preliminarily-captured image obtained by preliminary imaging executed prior to the main imaging on the subject and a main captured image obtained by the main imaging.

11. The medical image diagnostic apparatus according to claim 1, wherein the medical image is an image for diagnostic imaging on the subject.

12. The medical image diagnostic apparatus according to claim 2, wherein the medical image is an image visually recognizable in a similar manner as a main captured image obtained by the main imaging on the subject.

13. The medical image diagnostic apparatus according to claim 1, wherein the medical image corresponds to one of a medical image to be generated by a single medical image diagnostic apparatus and a fusion image obtained by combining a plurality of images to be generated by a plurality of medical image diagnostic apparatuses.

14. The medical image diagnostic apparatus according to claim 1, wherein the medical image corresponds to at least one of a plurality of sections in the subject.

15. The medical image diagnostic apparatus according to claim 1, wherein the medical image is an image obtained by visualizing anatomical information on the subject.

16. The medical image diagnostic apparatus according to claim 2, wherein the processing circuitry causes the display to display an imaging range for the main imaging by superimposing the imaging range on the medical image.

17. The medical image diagnostic apparatus according to claim 1, wherein the processing circuitry determines a posture state of the subject by applying the medical image to a posture output learned model trained to output a posture of the subject by input of the medical image.

18. The medical image diagnostic apparatus according to claim 17, wherein the posture state is a body position of the subject and/or an angle of the subject in a craniocaudal direction.

19. The medical image diagnostic apparatus according to claim 17, wherein the processing circuitry causes the display to display the posture state by superimposing the posture state on the optical image.

20. A medical image processing method comprising:
acquiring an optical image of a subject captured by an optical imaging device and/or skeleton information about the subject;
generating a medical image related to the subject based on the optical image and/or the skeleton information; and
presenting the medical image.
